(19) United States
(12) Patent Application Publication
(10) Pub. No.: US 2011/0245091 A1
(43) Pub. Date: Oct. 6, 2011

(54) REACTION PROGRESS ASSAY FOR SCREENING BIOLOGICAL ACTIVITY OF ENZYMES

(76) Inventor: Valeri Golovlev, Oak Ridge, TN (US)

(21) Appl. No.: 13/070,826

(22) Filed: Mar. 24, 2011

Related U.S. Application Data
(60) Provisional application No. 61/320,363, filed on Apr. 2, 2010.

(51) Int. Cl. C40B 30/00; C12Q 1/00; C12Q 1/48; C12Q 1/66

(52) U.S. Cl. 506/7; 435/4; 435/15; 435/8

(57) ABSTRACT

The present invention relates to assays for measurement activity of enzymes acting to produce products by consuming ATP or dATP substrate. The assays can also be used to identify and screen for substances that modulate the activity of ATP- and dATP-dependent enzymes.

The present application claims priority to U.S. Provisional Application Ser. No. 61/320,363 filed on Apr. 2, 2010

### FIELD OF THE INVENTION

This invention relates to assay for continuous monitoring enzymatic reactions and quantifying activity of enzymes generally related to drug discovery.

### BACKGROUND OF THE INVENTION

The disclosed screening assay is based, in part, on the discovery that continuous monitoring of substrate molecules consumed in biochemical reaction catalyzed by certain types of enzymes provides a superior technique for measurement biological activity of the respective enzymes. The said substrate molecules comprising ATP, GTP, CTP, UTP, dATP, dGTP, dCTP, dTTP their derivatives and other molecules and co-factors that are required for completion enzymatic reaction of interest. Enzymes may include viral, microbial and other polymerases, protein kinases, ATPases, topoisomerases, helicases, synthases and other proteins and enzymes that are targets in drug discovery. The measurement of the biological activity of enzyme of interest (target enzyme) in presence of various chemical compounds can be used for identifying compounds that modulate biological activity of said target enzyme either by increasing enzyme activity, referred herein as activation, or by decreasing target enzyme activity, referred herein as inhibition.

Enzymes which facilitate the use of energy-reach molecules such as NTP, dNTP, and derivatives thereof in various biochemical reactions are often fundamental to the biochemical function of many organisms and viruses.

In one embodiment of the method of present invention this group of enzymes includes, for example, terminal transferases, RNA and DNA polymerases. It is well recognized in the art that inhibitors or activators of these enzymes might be new classes of therapeutic or preventative compounds, particularly against cancer and viral diseases. A common technique for measurement activity of this group of enzymes can be illustrated by U.S. Pat. No. 6,100,028, incorporated herein by reference. Polymerase activity assay typically involve extension of DNA or RNA primer in presence of labeled nucleotides. The extension reaction is carried over a predetermined period of time, then the reaction is terminated and the reaction product, i.e., extended RNA or DNA primer, is measured often by radioactive or fluorescent detection. The amount of detected product is used as the measure of the enzyme activity. The method disclosed in U.S. Pat. No. 6,100,028 is particular sensitive when a radioactive labeling is used. For detection of the reaction product by that method a separation step is required that is often carried out either by gel electrophoresis or by binding the extended product to a membrane. Yet, the need for separation step hampers the use of the method in high-throughput screening assays. Therefore, there is unmet need for a better method for screening activity of terminal transferases, RNA and DNA polymerases and addressing that need is the aim of the method of present invention.

In another embodiment of the method of present invention a group of enzymes includes transferases, which catalyze the transfer of chemical groups between molecules, and more specifically, a subgroup of transferases called protein kinases.

Protein kinases are enzymes which catalyze the transfer of phosphorous from adenosine triphosphate (ATP), or guanosine triphosphate (GTP) to the targeted protein to yield a phosphorylated protein and adenosine diphosphate (ADP) or guanosine diphosphate (GDP), respectively. ATP or GTP is first hydrolyzed to form ADP or GDP and inorganic phosphate. The inorganic phosphate is then attached to the targeted protein. The protein substrate which is targeted by kinases may be a structural protein, found in membrane material such as a cell wall, or another enzyme which is a functional protein.

Due to their physiological relevance, variety and ubiquitousness, protein kinases have become one of the most important and widely studied family of enzymes in biochemical and medical research. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis.

Traditional methods of measuring the state of phosphorylation of cellular proteins are based on incorporation of radioactive .sup.32P-orthophosphate as can be illustrated by U.S. Pat. No. 6,066,462, incorporated herein by reference. The .sup.32P-phosphorylated proteins are separated on a gel and subsequently visualized using a phospho-imager. Alternatively, phosphorylated tyrosine residues may be bound via binding of radiolabeled anti-phosphotyrosine antibodies and detected by immunoassays, for example immunoprecipitation or blotting. Since these methods need to detect radioisotopes, they are time-consuming and also, owing to the safety aspects involved in the handling of radioactive substances, not suitable for high throughput screening (HTS).

In more recent methods, the radioactive immunoassays are replaced with ELISAs (enzyme-linked immunosorbent assays). These methods use purified substrate proteins or synthetic peptide substrates which have been immobilized to a substrate surface. After a kinase action, the extent of phosphorylation is quantified by the binding of anti-phosphotyrosine antibodies coupled to an enhancer enzyme such as peroxidases, for example, to the phosphorylated immobilized substrates as can be illustrated by U.S. Pat. No. 6,203,994, incorporated herein by reference. These antibodies, however, have the property of recognizing not only phosphoserine but also the neighboring amino acids as epitope. It is known, however, that kinases function very substrate-specifically and that substrate sequences can differ greatly. Therefore, anti-phosphoserine antibodies cannot be used as generic reagents.

Perkin Elmer (Wallac) provide an assay for tyrosine kinases, which is based on time-resolved fluorescence and an energy transfer from europium chelates to allophycocyanine (see also EP 929 810). Here too, the process is limited essentially to tyrosine kinases, due to the use of antibodies.

It is appreciated that, in addition to enzymes described herein above, other groups of enzyme can be practiced with the method of present invention including, but not limited to ATPases, topoisomerases, helicases, and synthases. The feature common to enzymes in the method of present invention is the catalytic activity in reactions that involve NTP and dNTP molecules and most preferably ATP, GTP, dATP, and dGTP.

Now considering ATPase and helicases as example, Human papillomaviruses (HPVs) are the causative agents of benign and malignant lesions of the epithelium. The ATPase and helicase activities of the highly conserved E1 protein of HPV are essential for viral DNA replication and pathogenesis and hence are considered valid antiviral targets. E1 is the replicative helicase of papillomaviruses. It binds cooperatively to the origin of replication in conjunction with the E2 protein. Formation of the E1-E2-origin complex involves not only the binding of both proteins to specific DNA elements in the origin but also a protein-protein interaction between the N-terminal transactivation domain of E2 and the helicase/ATPase domain of E1. Recently a new class of small-molecule inhibitors of HPV DNA replication has been reported that bind to the transactivation domain of E2 and prevent its interaction with E1. Both ATPases and helicases, when active, catalyze the decomposition of adenosine triphosphate (ATP) into adenosine diphosphate and therefore, the activity of respective enzymes can be detected by monitoring the amount of ATP in reaction solution according to the method of present invention.

Other enzymatic reactions that can be used with the method of present invention include, but not limited to, reactions catalyzed by adenylate cyclase which uses ATP to produce the second messenger molecule cyclic AMP and also various enzymes participating in metabolic reactions leading to production or consumption energy-reach molecules, such as purines and pyrimidines and its derivatives (ATP, GTP, CTP, dATP, dGTP, dCTP, etc.), and most preferably including, but not limited to enzymatic reactions that produce or consume ATP and its derivatives, GTP and its derivatives, dATP and its derivatives, dGTP and its derivatives, and so forth. The existing methods to study such enzymatic reaction often are based on detection of radioactively-labeled probes and therefore require handling hazardous materials. Known assay of the previous art often are represented by end-point assay, which are time consuming, labor-intensive, and have a drawback of low accuracy and reproducibility.

Accordingly, all methods in the art have one or more deficiency. Some require the use of radioisotopes for labeling causing the safety concerns. Some others require separation and washing steps and are time-consuming. Yet, some others are lacking the sensitivity, or requiring expensive reagents.

An ideal enzyme assay system should have: a) high throughput; b) high sensitivity; and c) yield reach quantitative information preferably from a single assay run.

### SUMMARY OF THE INVENTION

To address the evolving needs in drug discovery new assays are needed that can be used with various target enzymes, and which assays are quantitative, robust, reliable, amenable for high-throughput, and can be performed at low cost in large and small drug discovery projects.

The method of present invention is based on performing two enzymatic reactions, the primary reaction catalyzed by a target enzyme, and the secondary detection reaction catalyzed by a detection enzyme, and performing both enzymatic reactions simultaneously in the same reaction volume. In the method of present invention the detection enzyme is selected from a group of enzymes which are capable of using the same substrate as the substrate required for the reaction catalyzed by target enzyme of the primary enzymatic reaction.

It is essential in the method of present invention to maintain conditions at which the primary enzymatic reaction consumes substantial portion of available substrate, e.g., at least 10% of the substrate available when the reaction started, and most preferably 90% or more of the substrate available when the reaction first started. It is also essential in the method of present invention to use detection enzymes that are capable of producing products and/or detectable physical effects and to provide conditions and means for continuous monitoring formation of said product(s) or detection of said physical affects produced by detection enzymatic reaction as the primary enzymatic reaction proceeds. The product and detectable effects may include but not limited to the light emission, the release of energy in the form of heat, the change of electric conductivity of the reaction media, as well as production of chemical species having specific light-absorption or luminescence property, etc.

In the most preferable embodiments the detection enzyme is selected from the group of luciferases, preferably from the group of EC 1.13.12.7 (firefly luciferase), EC 1.13.12.5 (renilla luciferase), and EC 1.14.14.3 (alkanal monooxygenase), and most preferably is the firefly luciferase (EC 1.13.12.7).

Considering now firefly luciferase as the detection enzyme, the method of present invention discloses assay for screening activity of NTP- and dNTP-dependent enzymes, and most preferably for screening ATP, dATP, GTP, and dGTP dependent target enzymes that include, but not limited to groups of terminal transferases, kinases, DNA- and RNA-polymerases.

The method of present invention also provides for a method for continuous monitoring enzymatic reaction and measurement enzyme activity comprising the steps: 1) contacting in single reaction volume the target enzyme, detection enzyme, substrate and other compounds that are required for the target and detection enzymatic reactions to proceed; 2) continuously measuring a signal vs. time generated by the detection enzymatic reaction, where said signal is proportional to the amount of substrate common to the detection and target enzymatic reaction; 3) calculating the reaction rate vs. time and constructing Michaelis-Menten plot for the target enzymatic reaction by processing recorded data for continuous monitoring of the product or physical effects generated by detection enzymatic reaction.

It is appreciated that in the method of present invention a set of data for constructing Michaelis-Menten plot for the target enzymatic reaction is acquired form a single assay run, without repeating measurements multiple times at various substrate concentration as it is commonly known in the art.

Furthermore, the present invention also provides for a method of screening for a substance that modulates the activity of an NTP- and dNTP-dependent enzymes. Said method comprising the steps of contacting the substrate, the target and detection enzymes in the presence or absence of the substance and by continuously measuring and recording signal vs. time generated by the detection enzymatic reaction. The recorded signal is generally proportional to the amount of NTP- and dNTP substrate vs. time and can be used to calculate reaction rate of the target enzyme vs. enzyme concentration as disclosed by the method of present invention. A data set is generated that can be used for quantitative determination of the target enzyme activity, the modulation effect of the substance and identification the substance modulation mechanisms such as competitive, non-competitive, and uncompetitive inhibition of the target enzyme.

In one embodiment, the acquired data set is used to construct a Michaelis-Menten plot or to implement data analysis techniques known in the art for determination target enzyme activity and measurement of the modulation effect of the respective substance.

Yet, in another embodiment the method of present invention discloses an alternative data analysis method for determination target enzyme activity and measurement the modulation effect of the respective substance.

Substances that may modulate activity of target enzyme can be screened individually or in parallel. An example of parallel screening is a high throughput drug screen of large libraries of chemicals as disclosed in U.S. Pat. Nos. 7,285,411 and 7,901,878 incorporated herein by reference. Such libraries of compounds can be screened to determine whether any members of the library have a desired activity and, if so, to identify the active species.

The foregoing and other advantages and features of the invention, and the manner in which the same are accomplished, will become more readily apparent upon consideration of the following detailed description of the invention taken in conjunction with the accompanying examples, which illustrate exemplary embodiments.

### BRIEF DESCRIPTION OF THE DRAWINGS

Drawing 1: Panel A illustrates luminescence signal vs. time acquired according to the method of present invention for enzymatic reaction catalyzed by HIV-I (Human Immunodeficiency Virus) reverse transcriptase (A1) with HIV-I transcriptase present and (A2) no HIV-I transcriptase present in sample. Panel B illustrates a Michaelis-Menten plot constructed from the luminescence data shown in Panel A.

Drawing 2 illustrates data analysis steps according to the method of present invention for measurement target enzyme activity (V.sub. max) and reaction constant (K.sub.max), including steps of determining and measuring crossing points y.sub. 1 and t.sub.1 of the trend line (TL) with the vertical axis (t=0) and the horizontal axis (y=1) and applying respective formulas for V.sub. max and K.sub.max as disclosed in the method of present invention.

Drawing 3: Panels (A-B) illustrate data analysis according to the method of present invention for determining inhibition mechanism of an inhibitory substance. In Panels (A-C) the plots from left to right correspond to increasing concentration of respective inhibitory substance, with the most left plot corresponding to no inhibitory substance present. A location of the crossing point of the trend line drawn to the plot with and without inhibitory substance is used to identify inhibitory mechanism of the substance. In this example, Panel A corresponds to non-competitive inhibition; Panel B corresponds to competitive inhibition; and Panel C corresponds to un-competitive inhibition. Panel D illustrates luminescence data for enzymatic reaction catalyzed by HIV-I reverse transcriptase with (D1) no inhibitory substance present; (D2) the inhibitor MT-4S is present at 1.2 nM and (D3) the MT-4S is present at 3.7 nM. The (D4) is a reference with no HIV-I transcriptase added. The crossing of trend lines for D1-D3 plots in a close proximity to the vertical axis indicate non-competitive mechanism of MT-4S inhibition, similar to that illustrated in panel A.

Drawing 4 illustrates the method of present invention for detection of weakly binding inhibitors for using the assay in Fragment-Based Drug Discovery (FBDD). Shown in this drawing is inhibition efficiency Q=1+[I]/K.sub.i for a set of a reference (no inhibitory compound) and 25 inhibitors of HIV-I reverse transcriptase measured by assay of the method of present invention. On this drawing the higher Q values corresponds to more efficient inhibitors and Q=1 corresponds to no inhibitory effect observed. Each compound was measured in triplicates and is shown on the plot by respective three data points. From data illustrated in this drawing the assay of present invention is able to detect compounds with K.sub.i up to 2,000 .mu.M as can be observed for, instance, for compound #13, # 15, and #23 (PubChem ID: CID546, CID790, and CID65059 respectively). Here, the K.sub.i has been calculated using known concentration of inhibitory compounds 0.5 mM in the assay reaction solution and the Q value of 1.25-1.27 found from experimental data for compound #13, #15, and #23. For FBDD applications it is often required to detect inhibitors with K.sub. I>100 .mu.M.

### DETAILED DESCRIPTION OF THE INVENTION

### Definitions

Throughout the specification and claims the following definitions shall apply:

Unless defined otherwise, all technical and scientific terms used above and throughout the text have the same meaning as commonly understood to one of ordinary skill in the art to which this invention belongs. Methods and materials similar or equivalent to those described herein can be used in the practice of the present invention, and exemplified suitable methods and materials are described below. For example, methods may be described which comprise more than two steps. In such methods, not all steps may be required to achieve a defined goal and the invention envisions the use of isolated steps to achieve these discrete goals. The disclosures of all publications, patent applications, patents and other references are incorporated in to herein by reference. In addition, the materials, methods, and examples are illustrative only and not intended to be limiting.

Terms that are not otherwise defined herein are used in accordance with their plain and/ordinary meaning.

“Enzyme” is meant to include various biological molecules having catalytic functions for transferring chemical groups between molecules to facilitate building up or tearing down others molecules.

“Substrate” is meant to include various organic molecules the reaction of which with some other molecules is catalyzed by enzyme.

The abbreviation “ATP” refers to adenosine-5′-triphosphate.

The abbreviation “GTP” refers to guanosine-5′-triphosphate.

The abbreviation “CTP” refers to cytidine-5′-triphosphate.

The abbreviation “UTP” refers to uridine-5′-triphosphate.

The abbreviation “dATP” refers to 2′-deoxyadenosine-5′-triphosphate.

The abbreviation “dGTP” refers to 2′-deoxyguanosine-5′-triphosphate.

The abbreviation “dCTP” refers to 2′-deoxycytidine-5′-triphosphate.

The abbreviation “dTTP” refers to 2′-deoxythymidine-5′-triphosphate.

“Nucleotide” is any of various compounds consisting of a nucleoside combined with a phosphate group and forming the basic constituent of DNA and RNA.

“Primer” is meant to include single stranded RNA and DNA molecules that can be extended by RNA and/or DNA polymerases.

“Template” is meant to include single stranded RNA and DNA molecules that can be hybridized to primer to facilitate primer extension by RNA and DNA polymerases.

The term “replication reaction” and “replication of nucleic acid” refers to an enzymatic biochemical reaction involving primer and template, in which reaction the primer and template have a homology and shorter primer molecules is extended according to the sequence of the template molecules. Examples of replication reaction include, but not limited to, the polymerase chain reaction (PCR).

The terms “polymerase” and “reverse transcriptase” refers to enzymes known in the art for carrying DNA and RNA replication reactions. Non-limiting examples include DNA-dependent DNA polymerases, DNA-dependent RNA polymerases, RNA-dependent DNA polymerases (e.g., transcriptases), and RNA-dependent RNA polymerases. Non-limiting examples of polymerases that may be of use include Thermatoga maritima DNA polymerase, AmplitaqFS™ DNA polymerase, Taquenase™ DNA polymerase, ThermoSequenase™, Taq DNA polymerase, Qbeta™ replicase, T4 DNA polymerase, and Thermus thernophilus DNA polymerase. Commercially available polymerases including Pwo DNA Polymerase from Boehringer Mannheim Biochemicals (Indianapolis, Ind.); Bst Polymerase from Bio-Rad Laboratories (Hercules, Calif.); IsoTherm™ DNA Polymerase from Epicentre Technologies (Madison, Wis.); Moloney Murine Leukemia Virus Reverse Transcriptase, Pfu DNA Polymerase, Avian Myeloblastosis Virus Reverse Transcriptase, Thermus flavus (Tfl) DNA Polymerase and Thermococcus litoralis (Tli) DNA Polymerase from Promega (Madison, Wis.); RAV2 Reverse Transcriptase, HIV-1 Reverse Transcriptase, T7 RNA Polymerase, T3 RNA Polymerase, SP6 RNA Polymerase, RNA Polymerase E. coli, Thermus aquaticus DNA Polymerase, T7 DNA Polymerase +/−3′.fwdarw.5′ exonuclease, Klenow Fragment of DNA Polymerase I, Thermus ‘ubiquitous’ DNA Polymerase, and DNA polymerase I from Amersham Pharmacia Biotech (Piscataway, N.J.), Method of using polymerases and compositions suitable for use in various methods are well known in the art (e.g., U.S. Pat. No. 7,141,370) and incorporated herein by reference in its entirety.

The term “viral polymerase” refers to a complete protein or fragment thereof naturally produced by a virus of interest or isolated by recombinant techniques, where said viral polymerase is capable of catalyzing extension of RNA or DNA primer at proper reaction conditions.

The term “enzymatic luminescence” or “luminescence” refers to one or more consecutive biochemical reactions involving at least one reaction of enzyme(s) and substrate(s) which said consecutive reactions are producing light as result of chemical or biochemical modification of the molecules involved.

The term “phosphorylation” refers to the introduction of a phosphate group to an organic molecule such as a polypeptide or to the formation (cyclization) of a ring connecting a phosphate group and a nucleoside in a nucleotide. A common cyclization forms cAMP and cGMP from ATP and GTP, respectively, by removing two phosphate groups from the nucleotide triphosphates and joining the free end of the remaining phosphate group to the sugar in the remaining nucleotide monophosphate.

The term “kinase” refers to an enzyme capable of phosphorylating certain polypeptides and proteins.

“NTP- and dNTP-dependent enzymes” refers to enzymes comprising of EC1.17.4.2 (ribonucleoside-triphosphate reductase), EC 1.2.1.30 (aryl-aldehyde dehydrogenase), EC 1.2.3.9 (aryl-aldehyde oxidase), EC 1.2.99.6 (Carboxylate reductase), EC 1.3.99.15 (Benzoyl-CoA reductase), EC 1.5.1.6 (formyltetrahydrofolate dehydrogenase), EC 1.6.2.2 (cytochrome-b5 reductase), EC 1.8.98.2 (sulfuredoxin), EC 1.8.99.2 (adenylyl-sulfate reductase), EC 1.13.12.5 (Renilla-luciferin 2-monooxygenase), EC 1.13.12.7 (Photinus-luciferin 4-monooxygenase (ATP-hydrolysing)), EC1.13.12.7 (Photinus-luciferin 4-monooxygenase (ATP-hydrolysing)), EC 1.13.12.8 (Watasenia-luciferin 2-monooxygenase), EC 1.14.14.3 (alkanal monooxygenase (FMN)), EC 1.14.19.1 (stearoyl-CoA 9-desaturase), EC 1.17.4.2 (ribonucleoside-triphosphate reductase), EC 1.18.6.1 (nitrogenase), EC 1.19.6.1 (nitrogenase (flavodoxin)), EC 2.1.1.B1 (protein-arginine Nomega-methyltransferase), EC 2.1.2.2 (phosphoribosylglycinamide formyltransferase), EC 2.3.1.B9 (tRNAMet cytidine acetyltransferase), EC 2.3.1.88 (peptide alpha-N-acetyltransferase), EC 2.3.3.8 (ATP citrate synthase), EC 2.4.2.9 (uracil phosphoribosyltransferase), EC 2.4.2.12 (nicotinamide phosphoribosyltransferase), EC 2.4.2.17 (ATP phosphoribosyltransferase), EC 2.5.1.6 (methionine adenosyltransferase), EC 2.5.1.17 (cob(I)yrinic acid a,c-diamide adenosyltransferase), EC 2.5.1.27 (adenylate dimethylallyltransferase), EC 2.6.99.1 (dATP(dGTP)-DNA purinetransferase), EC 2.7.1.1 (hexokinase), EC 2.7.1.3 (ketohexokinase), EC 2.7.1.4 (fructokinase), EC 2.7.1.5 (rhamnulokinase), EC 2.7.1.6 (galactokinase), EC 2.7.1.7 (mannokinase), EC 2.7.1.8 (glucosamine kinase), EC 2.7.1.10 (phosphoglucokinase), EC 2.7.1.811 (pantoate kinase), EC 2.7.1.12 (gluconokinase), EC 2.7.1.13 (dehydrogluconokinase), EC 2.7.1.14 (sedoheptulokinase), EC 2.7.1.15 (ribokinase), EC 2.7.1.16 (ribulokinase), EC 2.7.1.17 (xylulokinase), EC 2.7.1.18 (phosphoribokinase), EC 2.7.1.19 (phosphoribulokinase), EC2.7.1.20 (adenosine kinase), EC 2.7.1.21 (thymidine kinase), EC 2.7.1.22 (ribosylnicotinamide kinase), EC 2.7.1.23 (NAD+ kinase), EC 2.7.1.24 (dephospho-CoA kinase), EC 2.7.1.25 (adenylyl-sulfate kinase), EC 2.7.1.26 (riboflavin kinase), EC 2.7.1.27 (erythritol kinase), EC 2.7.1.28 (triokinase), EC 2.7.1.29 (glycerone kinase), EC 2.7.1.30 (glycerol kinase), EC 2.7.1.31 (glycerate kinase), EC 2.7.1.32 (choline kinase), EC 2.7.1.33 (pantothenate kinase), EC 2.7.1.34 (pantetheine kinase), EC 2.7.1.35 (pyridoxal kinase), EC 2.7.1.36 (mevalonate kinase), EC 2.7.1.37 (ATP:protein phosphotransferase), homoserine kinase), EC 2.7.1.39 (homoserine kinase), EC 2.7.1.40 (pyruvate kinase), EC 2.7.1.43 (glucuronokinase), EC 2.7.1.44 (galacturonokinase), EC 2.7.1.45 (2-dehydro-3-deoxygluconokinase), EC 2.7.1.46 (L-arabinokinase), EC 2.7.1.47 (D-ribulokinase), EC 2.7.1.48 (uridine kinase), EC 2.7.1.49 (hydroxymethylpyrimidine kinase), EC 2.7.1.50 (hydroxyethylthiazole kinase), EC 2.7.1.51 (L-fuculokinase), EC 2.7.1.52 (fucokinase), EC 2.7.1.53 (L-xylulokinase), EC 2.7.1.54 (D-arabinokinase), EC 2.7.1.55 (allose kinase), EC 2.7.1.56 (1-phosphofructokinase), EC 2.7.1.58 (2-dehydro-3-deoxygalactonokinase), EC 2.7.1.59 (N-acetylglucosamine kinase), EC 2.7.1.60 (N-acylmannosamine kinase), EC 2.7.1.63 (polyphosphate-glucose phosphotransferase), EC 2.7.1.64 (inositol 3-kinase), EC 2.7.1.65 (scyllo-inosamine 4-kinase), EC 2.7.1.66 (undecaprenol kinase), EC 2.7.1.67 (1-phosphatidylinositol 4-kinase), EC 2.7.1.68 (1-phosphatidylinositol-4-phosphate 5-kinase), EC 2.7.1.71 (shikimate kinase), EC 2.7.1.72 (streptomycin 6-kinase), EC 2.7.1.73 (inosine kinase), EC 2.7.1.74 (deoxycytidine kinase), EC 2.7.1.76 (deoxyadenosine kinase), EC 2.7.1.77 (nucleoside phosphotransferase), EC 2.7.1.78 (polynucleotide 5′-hydroxyl-kinase), EC2.7.1.78 (polynucleotide 5′-hydroxyl-kinase), EC 2.7.1.82 (ethanolamine kinase), EC 2.7.1.83 (pseudouridine kinase), EC 2.7.1.84 (alkylglycerone kinase), EC 2.7.1.85 (beta-glucoside kinase), EC 2.7.1.86 (NADH kinase), EC 2.7.1.87 (streptomycin 3″-kinase), EC 2.7.1.88 (dihydrostreptomycin-6-phosphate 3′ alpha-kinase), EC 2.7.1.89 (thiamine kinase), EC 2.7.1.91 (sphinganine kinase), EC 2.7.1.92 (5-dehydro-2-deoxygluconokinase), EC 2.7.1.93 (alkylglycerol kinase), EC 2.7.1.94 (acylglycerol kinase), EC 2.7.1.95 (kanamycin kinase), EC 2.7.1.100 (S-methyl-5-thioribose kinase), EC 2.7.1.101 (tagatose kinase), EC 2.7.1.102 (hamamelose kinase), EC 2.7.1.103 (viomycin kinase), EC 2.7.1.105 (6-phosphofructo-2-kinase), EC 2.7.1.107 (diacylglycerol kinase), EC2.7.1.113 (deoxyguanosine kinase), EC 2.7.1.119 (hygromycin-B 7″-O-kinase), EC 2.7.1.127 (inositol-trisphosphate 3-kinase), EC 2.7.1.130 (tetraacyldisaccharide 4′-kinase), EC 2.7.1.134 (inositol-tetrakisphosphate 1-kinase), EC 2.7.1.136 (macrolide 2′-kinase), EC 2.7.1.137 (phosphatidylinositol 3-kinase), EC 2.7.1.138 (ceramide kinase), EC 2.7.1.140 (inositol-tetrakisphosphate 5-kinase), EC 2.7.1.144 (tagatose-6-phosphate kinase), EC 2.7.1.145 (deoxynucleoside kinase), EC 2.7.1.148 (4-(cytidine 5′-diphospho)-2-C-methyl-D-erythritol kinase), EC 2.7.1.149 (1-phosphatidylinositol-5-phosphate 4-kinase), EC 2.7.1.150 (1-phosphatidylinositol-3-phosphate 5-kinase), EC 2.7.1.151 (inositol-polyphosphate i), EC 2.7.1.153 (phosphatidylinositol-4,5-bisphosphate 3-kinase), EC 2.7.1.154 (phosphatidylinositol-4-phosphate 3-kinase), EC 2.7.1.156 (adenosylcobinamide kinase), EC 2.7.1.157 (N-acetylgalactosamine kinase), EC 2.7.1.158 (inositol-pentakisphosphate 2-kinase), EC 2.7.1.159 (inositol-1,3,4-trisphosphate 5/6-kinase), EC 2.7.1.161 (CTP-dependent riboflavin kinase), EC 2.7.1.162 (N-acetylhexosamine 1-kinase), EC 2.7.1.163 (hygromycin B 4-O-kinase), EC 2.7.1.164 (O-phosphoseryl-tRNASec kinase), EC 2.7.1.165 (glycerate 2-kinase), EC 2.7.1.166 (3-deoxy-D-manno-octulosonic acid kinase), EC 2.7.1.167 (D-glycero-beta-D-manno-heptose-7-phosphate kinase), EC 2.7.1.168 (D-glycero-alpha-D-manno-heptose-7-phosphate kinase), EC 2.7.2.1 (acetate kinase), EC 2.7.2.2 (carbamate kinase), EC 2.7.2.3 (phosphoglycerate kinase), EC 2.7.2.4 (aspartate kinase), EC 2.7.2.6 (formate kinase), EC 2.7.2.7 (butyrate kinase), EC 2.7.2.8 (acetylglutamate kinase), EC 2.7.2.10 (phosphoglycerate kinase (GTP)), EC 2.7.2.11 (glutamate 5-kinase), EC 2.7.2.13 (glutamate 1-kinase), EC 2.7.2.14 (branched-chain-fatty-acid kinase), EC 2.7.2.15 (propionate kinase), EC 2.7.3.1 (guanidinoacetate kinase), EC 2.7.3.2 (creatine kinase), EC 2.7.3.3 (arginine kinase), EC 2.7.3.4 (taurocyamine kinase), EC 2.7.3.5 (lombricine kinase), EC 2.7.3.6 (hypotaurocyamine kinase), EC 2.7.3.7 (opheline kinase), EC 2.7.3.10 (agmatine kinase), EC 2.7.4.1 (polyphosphate kinase), EC 2.7.4.B1 (yeast UMP kinase), EC 2.7.4.2 (phosphomevalonate kinase), EC 2.7.4.3 (adenylate kinase), EC 2.7.4.4 (nucleoside-phosphate kinase), EC 2.7.4.6 (nucleoside-diphosphate kinase), EC 2.7.4.7 (phosphomethylpyrimidine kinase), EC 2.7.4.8 (guanylate kinase), EC2.7.4.9 (dTMP kinase), EC 2.7.4.10 (nucleoside-triphosphate-adenylate kinase), EC 2.7.4.11 ((deoxy)adenylate kinase), EC 2.7.4.12 (T2-induced deoxynucleotide kinase), EC 2.7.4.13 ((deoxy)nucleoside-phosphate kinase), EC 2.7.4.14 (cytidylate kinase), EC 2.7.4.15 (thiamine-diphosphate kinase), EC 2.7.4.16 (thiamine-phosphate kinase), EC 2.7.4.18 (farnesyl-diphosphate kinase), EC 2.7.4.19 (5-methyldeoxycytidine-5′-phosphate kinase), EC 2.7.4.21 (inositol-hexakisphosphate kinase), EC 2.7.4.22 (UMP kinase), EC 2.7.4.23 (ribose 1,5-bisphosphate phosphokinase), EC 2.7.4.24 (diphosphoinositol-pentakisphosphate kinase), EC 2.7.6.1 (ribose-phosphate diphosphokinase), EC 2.7.6.2 (thiamine diphosphokinase), EC 2.7.6.3 (2-amino-4-hydroxy-6-hydroxymethyldihydropteridine diphosphokinase), EC 2.7.6.4 (nucleotide diphosphokinase), EC 2.7.6.5 (GTP diphosphokinase), EC 2.7.7.1 (nicotinamide-nucleotide adenylyltransferase), EC 2.7.7.2 (FAD synthetase), EC 2.7.7.B3 (thiamine diphosphate adenylyl transferase), EC 2.7.7.4 (sulfate adenylyltransferase), EC 2.7.7.B4 (adenylyltransferase UBA4), EC 2.7.7.B5 (adenylyltransferase Thil), EC 2.7.7.6 (DNA-directed RNA polymerase), EC 2.7.7.7 (DNA-directed DNA polymerase), EC 2.7.7.8 (polyribonucleotide nucleotidyltransferase), EC 2.7.7.9 (UTP-glucose-1-phosphate uridylyltransferase), EC 2.7.7.13 (mannose-1-phosphate guanylyltransferase), EC 2.7.7.18 (nicotinate-nucleotide adenylyltransferase), EC 2.7.7.19 (polynucleotide adenylyltransferase), EC 2.7.7.21 (tRNA cytidylyltransferase), EC 2.7.7.24 (glucose-1-phosphate thymidylyltransferase), EC 2.7.7.25 (tRNA adenylyltransferase), EC 2.7.7.31 (DNA nucleotidylexotransferase), EC 2.7.7.30 (fucose-1-phosphate guanylyltransferase), EC 2.7.7.42 ([glutamate-ammonia-ligase] adenylyltransferase), EC 2.7.7.46 (gentamicin 2″-nucleotidyltransferase), EC 2.7.7.47 (streptomycin 3″-adenylyltransferase), EC 2.7.7.48 (RNA-directed RNA polymerase), EC 2.7.7.49 (RNA-directed DNA polymerase), EC 2.7.7.52 (RNA uridylyltransferase), EC 2.7.7.53 (ATP adenylyltransferase), EC 2.7.7.54 (phenylalanine adenylyltransferase), EC 2.7.7.55 (anthranilate adenylyltransferase), EC 2.7.7.58 ((2,3-dihydroxybenzoyl)adenylate synthase), EC 2.7.7.60 (2-C-methyl-D-erythritol 4-phosphate cytidylyltransferase), EC 2.7.7.61 (citrate lyase holo-[acyl-carrier protein] synthase), EC 2.7.7.63 (lipoate-protein ligase), EC 2.7.7.70 (D-glycero-beta-D-manno-heptose 1-phosphate adenylyltransferase), EC 2.7.8.25 (triphosphoribosyl-dephospho-CoA synthase), EC 2.7.9.1 (pyruvate, phosphate dikinase), EC 2.7.9.2 (pyruvate, water dikinase), EC 2.7.9.3 (selenide, water dikinase), EC 2.7.9.4 (alpha-glucan, water dikinase), EC 2.7.9.5 (phosphoglucan, water dikinase), EC 2.7.10.1 (receptor protein-tyrosine kinase), EC 2.7.10.2 (non-specific protein-tyrosine kinase), EC 2.7.11.1 (non-specific serine/threonine protein kinase), EC 2.7.11.2 ([pyruvate dehydrogenase (acetyl-transferring)] kinase), EC 2.7.11.3 (dephospho-[reductase kinase] kinase), EC 2.7.11.4 ([3-methyl-2-oxobutanoate dehydrogenase (acetyl-transferring)] kinase), EC 2.7.11.5 ([Isocitrate dehydrogenase (NADP+)] kinase), EC 2.7.11.6 ([tyrosine 3-monooxygenase] kinase), EC 2.7.11.7 (myosin-heavy-chain kinase), EC 2.7.11.8 (Fas-activated serine/threonine kinase), EC 2.7.11.9 (Goodpasture-antigen-binding protein kinase), EC 2.7.11.10 (IkappaB kinase), EC 2.7.11.11 (cAMP-dependent protein kinase), EC 2.7.11.12 (cGMP-dependent protein kinase), EC 2.7.11.13 (protein kinase C), EC 2.7.11.14 (rhodopsin kinase), EC 2.7.11.15 (beta-adrenergic-receptor kinase), EC 2.7.11.16 (G-protein-coupled receptor kinase), EC 2.7.11.17 (Ca2+/calmodulin-dependent protein kinase), EC 2.7.11.18 (myosin-light-chain kinase), EC 2.7.11.19 (phosphorylase kinase), EC 2.7.11.20 (elongation factor 2 kinase), EC 2.7.11.21 (polo kinase), EC 2.7.11.22 (cyclin-dependent kinase), EC 2.7.11.23 ([RNA-polymerase]-subunit kinase), EC 2.7.11.24 (mitogen-activated protein kinase), EC 2.7.11.25 (mitogen-activated protein kinase kinase kinase), EC 2.7.11.29 (low-density-lipoprotein receptor kinase), EC 2.7.11.30 (receptor protein serine/threonine kinase), EC 2.7.11.31 ([hydroxymethylglutaryl-CoA reductase (NADPH)] kinase), EC 2.7.12.1 (dual-specificity kinase), EC 2.7.12.2 (mitogen-activated protein kinase kinase), EC 2.7.13.1 (protein-histidine pros-kinase), EC 2.7.13.2 (protein-histidine tele-kinase), EC 2.7.13.3 (histidine kinase), EC 3.6.1.7 (acylphosphatase), EC 6.3.2.19 (Ubiquitin-protein ligase), EC 6.3.2.20 (Indoleacetate-lysine synthetase), EC 6.3.2.21 (ubiquitin-calmodulin ligase), EC 6.3.2.23 (homoglutathione synthase), EC 6.3.2.24 (tyrosine-arginine ligase), EC 6.3.2.25 (Tubulin-tyrosine ligase), EC 6.3.2.26 (N-(5-amino-5-carboxypentanoyl)-L-cysteinyl-D-valine synthase), EC 6.3.2.27 (aerobactin synthase), EC 6.3.2.28 (L-amino-acid alpha-ligase), EC 6.3.2.29 (cyanophycin synthase (L-aspartate-adding)), EC 6.3.2.30 (cyanophycin synthase (L-arginine-adding)), EC 6.3.2.31 (coenzyme F420-0:L-glutamate ligase), EC 6.3.2.32 (coenzyme gamma-F420-2:alpha-L-glutamate ligase), EC 6.3.2.33 (tetrahydrosarcinapterin synthase), EC 6.3.3.1 (phosphoribosylformylglycinamidine cyclo-ligase), EC 6.3.3.2 (5-Formyltetrahydrofolate cyclo-ligase), EC 6.3.3.3 (Dethiobiotin synthase), EC 6.3.3.4 ((carboxyethyl)arginine beta-lactam-synthase), EC 6.3.4.1 (GMP synthase), EC 6.3.4.B1 (tRNAlle-2-agmatinylcytidine synthetase), EC 6.3.4.2 (CTP synthase), EC 6.3.4.B2 (tRNAlle-2-agmatinylcytidine synthetase), EC 6.3.4.3 (formate-tetrahydrofolate ligase), EC 6.3.4.4 (Adenylosuccinate synthase), EC 6.3.4.5 (Argininosuccinate synthase), EC 6.3.4.6 (Urea carboxylase), EC 6.3.4.7 (Ribose-5-phosphate-ammonia ligase), EC 6.3.4.8 (Imidazoleacetate-phosphoribosyldiphosphate ligase), EC 6.3.4.9 (Biotin-[methylmalonyl-CoA-carboxytransferase] ligase), EC 6.3.4.10 (Biotin-[propionyl-CoA-carboxylase (ATP-hydrolysing)] ligase), EC 6.3.4.11 (Biotin-[methylcrotonoyl-CoA-carboxylase] ligase), EC 6.3.4.12 (Glutamate-methylamine ligase), EC 6.3.4.13 (phosphoribosylamine-glycine ligase), EC 6.3.4.14 (Biotin carboxylase), EC 6.3.4.15 (Biotin-[acetyl-CoA-carboxylase] ligase), EC 6.3.4.16 (Carbamoyl-phosphate synthase (ammonia)), EC 6.3.4.17 (Formate-dihydrofolate ligase), EC 6.3.4.18 (5-(carboxyamino)imidazole ribonucleotide synthase), EC 6.3.5.1 (NAD+ synthase (glutamine-hydrolysing)), EC 6.3.5.2 (GMP synthase (glutamine-hydrolysing)), EC 6.3.5.3 (phosphoribosylformylglycinamidine synthase), EC 6.3.5.4 (Asparagine synthase (glutamine-hydrolysing)), EC 6.3.5.5 (Carbamoyl-phosphate synthase (glutamine-hydrolysing)), EC 6.3.5.6 (asparaginyl-tRNA synthase (glutamine-hydrolysing)), EC 6.3.5.7 (glutaminyl-tRNA synthase (glutamine-hydrolysing)), EC 6.3.5.9 (hydrogenobyrinic acid a,c-diamide synthase (glutamine-hydrolysing)), EC 6.3.5.10 (adenosylcobyric acid synthase (glutamine-hydrolysing)), EC 6.4.1.1 (pyruvate carboxylase), EC 6.4.1.2 (acetyl-CoA carboxylase), EC 6.4.1.3 (propionyl-CoA carboxylase), EC 6.4.1.4 (methylcrotonoyl-CoA carboxylase), EC 6.4.1.5 (geranoyl-CoA carboxylase), EC 6.4.1.6 (acetone carboxylase), EC 6.4.1.7 (2-oxoglutarate carboxylase), EC 6.5.1.1 (DNA ligase (ATP)), EC 6.5.1.2 (DNA ligase), EC 6.5.1.3 (RNA ligase (ATP)), EC 6.5.1.4 (RNA-3′-phosphate cyclase), EC 6.6.1.1 (magnesium chelatase), and EC 6.6.1.2 (cobaltochelatase)

It must be noted that as used herein and in the appended claims, the singular forms “a”, “and”, and “the” include plural referents unless the context clearly dictates otherwise. Thus, for example, reference to “a target enzyme” may include plurality of enzymes from various classes including polymerases, protein kinases, ATPases, topoisomerases, helicases, synthases and equivalents thereof known to those skilled in the art, and so forth.

### DESCRIPTION OF ILLUSTRATIVE EMBODIMENTS

In accordance with the objects outlined above, the present invention provides methods, instruments, kits, and data analysis techniques for detection of the progress of enzymatic reaction of interest in presence or absence of one or more chemical compound that may modulate said enzymatic reaction.

The method of present invention is based, in part, on the discovery that continuous monitoring of substrate molecules consumed in biochemical reaction catalyzed by certain types of enzymes provides a superior technique for measurement biological activity of the respective enzymes.

The method of present invention is essentially based on performing primary (target) and secondary (detection) enzymatic reactions simultaneously in the same reaction volume. The primary enzymatic reaction is catalyzed by a target enzyme preferably selected from a group of NTP and dNTP-dependent enzymes. As the primary enzymatic reaction progress, the respective NTP or dNTP substrate is consumed in the reaction of producing a product of the primary enzymatic reaction.

The secondary enzymatic reaction employs a detection enzyme selected for the ability to use the same NTP or dNTP substrate as the primary enzymatic reaction, and where said detection enzymatic reaction produces a detectable product or physical effect that can be used for quantitative measurement vs. time of the amount of NTP or dNTP substrate available for the primary and secondary enzymatic reactions as both reactions proceed.

In most preferable embodiments the detection enzymatic reaction is a luminogenic (e.g., enzymatic luminescence) reaction that emits light and where the light intensity is proportional to the amount of NTP and dNTP substrate present in the reaction volume.

Now considering illustrative embodiments provided herein by way of illustration and not by way of limitation, in one embodiment of the method of present invention for screening anticancer protein kinase inhibitors, two enzymes are required, i.e., a target kinase either serine, or equally acceptable, tyrosine kinase (EC 2.7.11.1) and firefly luciferase (EC 1.13.12.7). Here a target kinase and luciferase have a common substrate ATP. The kinase consumes ATP in the process of phosphorylation of a specific substrate protein or polypeptide.

Concurrently and at the same time, the luciferase is oxidizing luciferin by consuming ATP and emitting luminescence photons, which can be detected by a photon counting detector, a luminometer. The amount of luciferase in assay is selected to be below a certain level such that most of ATP substrate is consumed by target enzyme, the kinase in this example. The decline of luminescence over time is observed and recorded as the ATP substrate is consumed predominantly by the target enzymatic reaction catalyzed by kinase. The rate of decline of luminescence signal is proportional to the activity of target enzyme, kinase, wherein the intensity of luminescence signal is proportional to the amount of ATP at the respective moments of time as the target and detection enzymatic reactions proceed. The recorded data, i.e., luminescence intensity vs. time therefore can be used to construct a plot representing the rate of target enzymatic reaction vs. substrate concentration (ATP). The plot, known in the art as the Michaelis-Menten plot can be used for characterization of activity of the target enzyme.

In another embodiment of the method of present invention for screening for inhibition of viral polymerases (e.g., Human immunodeficiency virus (HIV), Hepatitis C Virus (HCV), Herpes Simplex Virus, mumps, etc.). The target polymerase and luciferase have a common substrate dATP. The rate of dATP consumption by the primary reaction (i.e., the reaction for DNA/RNA extension by a viral polymerase) in the presence of polymerase inhibitors of interest can be detected by monitoring luminescence intensity vs. time.

The amount of luciferase in assay is selected to be below a certain level such that most of dATP substrate is consumed by a target enzyme. The decline of luminescence over time is observed and recorded as the dATP substrate is consumed predominantly by enzymatic reaction catalyzed by a viral polymerase. The rate of decline of luminescence signal is proportional to the activity of target enzyme, polymerase, wherein the intensity of luminescence signal is proportional to the amount of dATP at the respective moments of time as the target and detection enzymatic reactions proceed. The recorded data, i.e., luminescence intensity vs. time subsequently can be used to construct a plot representing the rate of target enzymatic reaction vs. substrate concentration (dATP). Once again, the plot, known in the art as the Michaelis-Menten plot can be used for characterization of activity of the target enzyme.

It is appreciated that in addition to the target enzyme, detection enzyme and primer the other reagents and components may be required to maintain activity of enzymes and support conditions for primary and secondary enzymatic reactions to proceeds. The additional reagents and components may include buffer, primers, templates, polypeptides and proteins on which target enzyme acting on, metal ions and co-factors known in the art and disclosed in U.S. Pat. Nos. 5,600,989; 6,066,462; 6,100,028; 7,070,921; 7,732,128; and 7,901,901 incorporated herein by reference.

It is appreciated that according to the method of present invention a non-luminogenic, e.g., fluorescent or colorimetric detection can be employed as well to detect a substrate concentration of the target enzymatic reaction vs. time. Examples of non-luminogenic detection include, but not limited to reactions which directly or indirectly measure the amount or presence of a substrate for the target reaction. For instance, a substrate for an enzyme may be modified to contain a fluorophore that emits light of a certain wavelength only after the enzyme reacts with the substrate and the fluorophore is contacted with (exposed to) light of a certain wavelength or range of wavelengths. In another example, a substrate for an enzyme may be modified to contain or may naturally carry out a chromophore that absorb light of a certain wavelength only after the enzyme reacts with the substrate and the chromophore is contacted with (exposed to) light of a certain wavelength or range of wavelengths.

One group of fluorophores consists of xanthene dyes and include the fluoresceins, rosamines and rhodamines. These compounds are commercially available with substituents on the phenyl group, which can be used as the site for bonding or as the bonding functionality. For example, amino and isothiocyanate substituted fluorescein compounds are available.

Another group of fluorophores are the naphthylamines, having an amino group in the alpha or beta position, usually alpha position. Included among the naphthylamino compounds are 1-dimethylaminonaphthyl-5-sulfonate, 1-anilino-8-napththalene sulfonate and 2-p-toluidinyl-6-naphthalene sulfonate. Some naphthalene compounds are found to have some non-specific binding to protein, so that their use requires employing an assay medium where the amount of protein is minimized. Other fluorophores are multidentate ligands that include nitrogen-containing macrocycles, which have conjugated ring systems with pi-electrons. These macrocycles may be optionally substituted, including substitution on bridging carbons or on nitrogens. Suitable macrocycles include derivatives of porphyrins, azaporphyrins, corrins, sapphyrins and porphycenes and other like macrocycles, which contain electrons that are extensively delocalized. The azaporphyrin derivatives include phthalocyanine, benzotriazaporphyrin and naphthalocyanine and their derivatives.

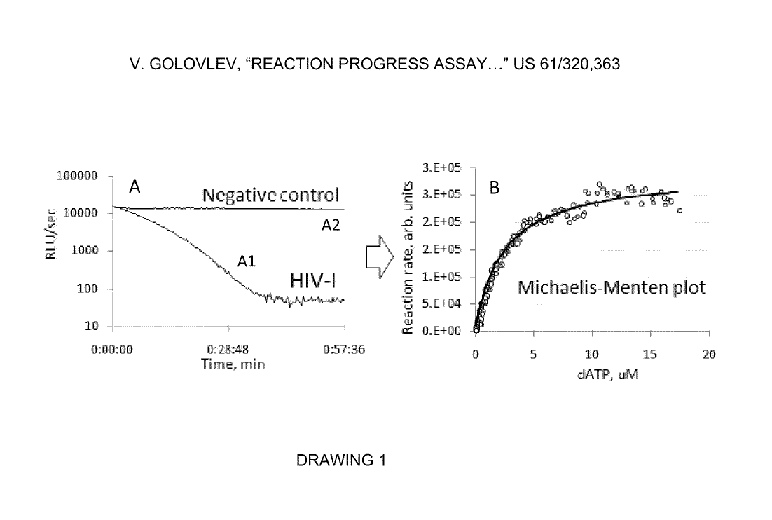

In most preferred embodiments, the intensity of light produced by luminogenic or, equally acceptable, non-luminogenic means for detecting substrate consumption by a target enzymatic reaction vs. time is proportional to the concentration of substrate at corresponding moments of time. Now considering luminescence detection as an example given by way of illustration, and not by way of limitation, the luminescence signal declines as the substrate is consumed. The time derivative, ∂ . . . l ∂t, of the luminescent signal, l(t), is proportional to the rate of substrate, S, consumption, ∂[s]l ∂t. With proper selection of experimental conditions, the first derivative of luminescence intensity over time, ∂l(t)l ∂t represents the rate of substrate consumption by the primary enzymatic reaction, such as protein phosphorylation and DNA/RNA extension in examples herein above. Indeed, due to the high sensitivity of photon-counting luminometers, the concentration of luciferase can be kept low compared with the concentration of the target enzyme of the primary enzymatic reaction. At such conditions, a plot of ∂ l(t)l ∂t at vs. l(t) represents the reaction rate of the target enzymatic reaction vs. substrate concentration, e.g., a Michaelis-Menten plot for the target enzyme as illustrated in FIG. 1.

The Michaelis-Menten plot subsequently can be used for determining the Michaelis-Menten constant K .sub. m, inhibition constant K.sub.i and for identifying the inhibitor binding mode as known in the art and disclosed, for instance by Zhang et al (Antimicrobial Agents and Chemotherapy, August 2006, p. 2772-2781), incorporated herein by reference.

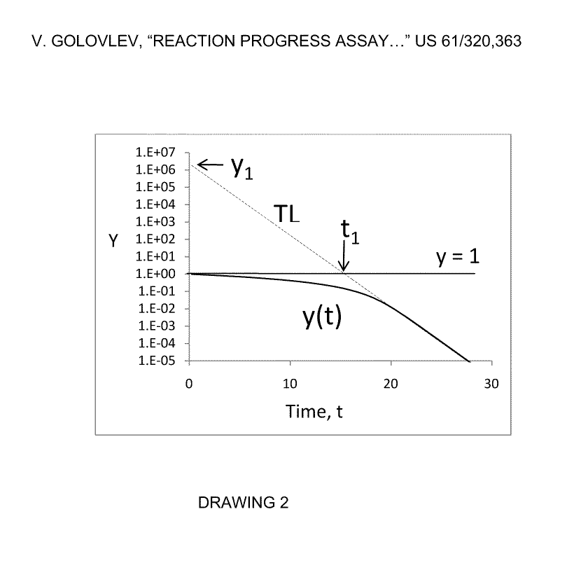

In another embodiment of the method of present invention the recorded data set of luminescent vs. time can be directly used to measure the activity of the target enzyme as illustrated in FIG. 2. First, a semi-logarithmic plot of luminescence intensity y(t)=l(t)/l.sub.0 vs. time is constructed, where l.sub.0=l(t=0). On said semi-log plot a trend line is constructed to approximate log (y(t)) at low luminescence intensity, where the decline of luminescence l(t) follows closely a simple exponential decline. The intersection points y.sub.1 and t.sub.1 of the trend line with the vertical (t=0) and horizontal (y=1) axis are measured as illustrated in FIG. 2. Michaelis-Menten parameters for the target enzyme, i.e., the maximum reaction rate V.sub.max and the reaction constant K.sub.m can be expressed using the y.sub.1 and t.sub.1 values and concentration of substrate So at t=0 according to formulas:

V.sub.max=S.sub.0/t.sub.1

and

K.sub.m=S.sub.0/log(y.sub.1)

It is appreciated that in presence of compound inhibiting target enzyme the formulas includes additional term [I]/K.sub.i, where [I] is the concentration of the inhibitory compound and K.sub. I is inhibition constant. In presence of inhibitory compound acting through competitive mechanism of inhibition:

V.sub.max=S.sub.0/t.sub.1

and

K.sub.m=S.sub.0/{log(y.sub.1)*(1+[I]/K.sub.i)}

For a compound acting through non-competitive mechanism:

K.sub.m=S.sub.0/log(y.sub.1)

and

V.sub.max={(1+[I]/K.sub.i)*S.sub.0}/t.sub.1

In accordance with this invention, the sample to be analyzed may contain enzyme having unknown activity V.sub.max. In the respective embodiment of the invention, the activity of enzyme present in a sample may be quantified by recording luminescence vs. time and by processing data as described hereinabove.

In an alternative embodiment, the sample may contain a substance whose inhibitory activity to the target enzyme is to be determined. This can be achieved by comprising the steps of contacting the substrate, the target and detection enzymes in the presence or absence of the substance and by continuously measuring and recording signal vs. time generated by the detection enzymatic reaction. The recorded signal is generally proportional to the amount of NTP- and dNTP substrate vs. time and can be used to calculate reaction rate of the target enzyme vs. enzyme concentration and construct Michaelis-Menten plot or by employing alternative methods for data analysis and identification inhibition parameters of the inhibitory substance, including construction of various plots known in the art: Lineweaver-Burk by plotting (∂ l(t)l ∂t)⁻¹ vs. (l(t))⁻¹; Woolf-Hanes by plotting l(t)*(∂ l(t)l ∂t)⁻¹ vs. l(t), and Eadie-Hofstee by plotting (∂ l(t)l ∂t) vs. l(t)*(∂ l(t)l ∂t)⁻¹ and using the respective plots for identification inhibition parameters and inhibition mechanism as disclosed by Atkinson and Nimmo (Biochemical Journal 149 (3): 775-777 (1975)) incorporated herein by reference.

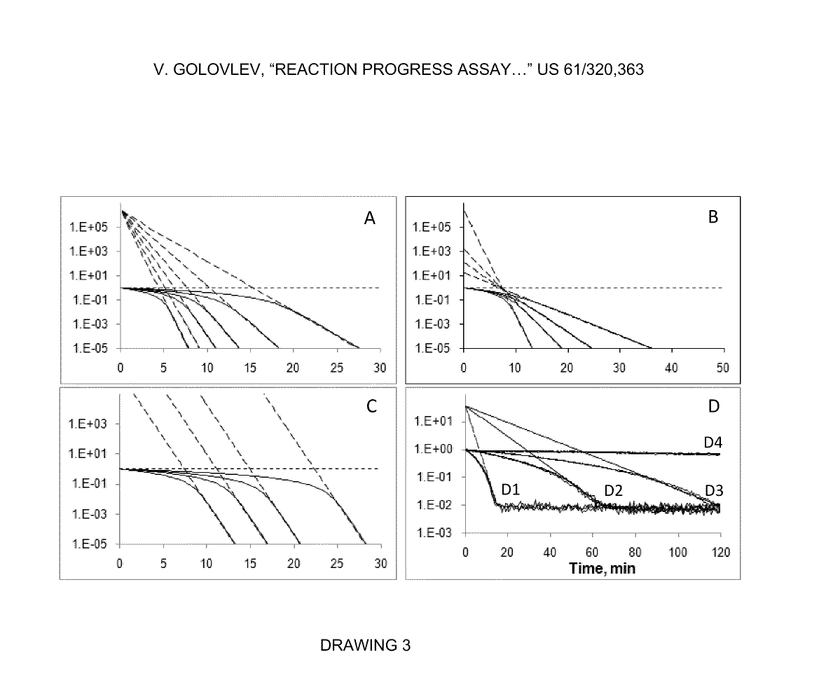

In another embodiment of the method of present invention the recorded data set of luminescent vs. time can be directly used to identify inhibition coefficient K.sub.i and inhibition mechanism of the inhibitory substance as illustrated in FIG. 3. A luminescence vs. time is recorded for two samples: one having inhibitory substance added and another without inhibitory substance present. For both samples a semi-logarithmic plot of luminescence intensity y(t)=l(t)/l.sub.0 vs. time is constructed, where l.sub.0=l(t=0). On said semi-log plot trend lines are constructed to approximate log(y(t)) for each sample at low luminescence intensity, where the decline of luminescence l(t) follows closely a simple exponential decline. The intersection points y.sub.1 and t.sub.1 of the trend lines with the vertical (t=0) and horizontal (y=1) axis are determined for the respective plots representing each sample. The K.sub.m and V.sub.max for each sample are measured as described hereinabove. For the inhibitory substance acting through competitive mechanism, the ratio of K.sub.m for the sample with inhibitory agent added to K.sub.m for the sample without inhibitory agent present is equal to (1+[I]/K.sub.i), where [I] is the concentration of the inhibitory agent and K.sub.i is the inhibition constant.

For an inhibitory substance acting through a non-competitive mechanism the ratio of V.sub.max for the sample without inhibitory agent added to the V.sub.max for the sample with inhibitory agent present is equal to (1+[I]/K.sub.i), where [I] is the concentration of the inhibitory agent and K.sub.i is inhibition constant.

In addition, an intersection point of the trend lines for samples with and without inhibitor agent is investigated. Here the intersection of the trend lines in close proximity to the horizontal axis (y=1) is indicative of a competitive mechanism of inhibition. Intersection of the trend lines in close proximity to the vertical axis (t=0) is indicative of a non-competitive mechanism and near parallel trend lines are indicative of uncompetitive inhibition mechanism of the inhibitory substance.

In various embodiments the assay of this invention can be used to identify compounds which, when present, may modulate activity of target enzyme. In certain embodiments, screens of the present invention utilize libraries of test compounds. As used herein, a “test compound” can be any chemical compound, for example, a macromolecule (e.g., a polypeptide, a protein complex, glycoprotein, or a nucleic acid) or a small molecule (e.g., an amino acid, a nucleotide, an organic or inorganic compound).

A test compound can have a formula weight of less than about 10,000 grams per mole, less than 5,000 grams per mole, less than 1,000 grams per mole, or less than about 500 grams per mole. The test compound can be naturally occurring (e.g., an herb or a natural product), synthetic, or can include both natural and synthetic components. Examples of test compounds include peptides, peptidomimetics (e.g., peptoids), amino acids, amino acid analogs, polynucleotides, polynucleotide analogs, nucleotides, nucleotide analogs, and organic or inorganic compounds (e.g., heteroorganic or organometallic compounds).

Test compounds can be screened individually or in parallel. An example of parallel screening is a high throughput drug screen of large libraries of chemicals. Such libraries of test compounds can be generated or purchased, e.g., from Chembridge Corp., San Diego, Calif. Libraries can be designed to cover a diverse range of compounds. For example, a library can include 500, 1000, 10,000, 50,000, or 100,000 or more unique compounds. Libraries of compounds can be screened to determine whether any members of the library have a desired activity and, if so, to identify the active species for further use in drug discovery.

The assay components and/or sample may be supported for contact by any material capable of providing such support. Suitable substrates may include microplates, PCR plates, biochips, and hybridization chambers, among others, where features such as microplate wells and microarray (i.e., biochip) sites may comprise assay sites. Suitable microplates are described in the following U.S. patent applications, which are incorporated herein by reference: Ser. No. 08/840,553, filed Apr. 14, 1997; and Ser. No. 09/478,819, filed Jan. 5, 2000. These microplates may include 96, 384, 1536, or other numbers of wells. These microplates also may include wells having small (.Itoreq.50 .mu.L) volumes, elevated bottoms, and/or frusto-conical shapes capable of matching a sensed volume.

### EXAMPLES

Now specific examples of the method of present invention are provided by way of illustration and not by way of limitation. An ordinary skilled artisan will recognize many various ways of practicing the method of present invention.

### Example 1

FIG. 1 shows an example of luminescence vs. time captured for the primary reaction of cDNA synthesis carried out by HIV-I RNA-dependent DNA polymerase (reverse transcriptase) in presence of MT-4S allosteric inhibitor (CAS 147127-20-6, U.S. Pat. No. 6,353,112, incorporated herein by reference). The reaction was set by adding: 16 .mu.L of RNAse-free water; 2 .mu.L of 10× reaction buffer (400 mM Tris-HCl, 60 mM MgCl.sub.2, 100 mM Dithiothreitol, 20 mM spermidine, pH 7.9 @ 25° C.); 2 .mu.L of template, polyuridylic acid at 1 .mu.g/.mu.L (Midland Certified Reagent Company, Cat. No. P-3004); 2 .mu.L of primer poly(A).sub.18 at 0.5 .mu.g/.mu.L (Polydeoxyadenylate, 18 base long purchased from Integrated DNA technologies); 20 .mu.M of dATP; 3 U of HIV-I (Worthington, Cat. No. LS05006); 3 .mu.L of ATP detection solution prepared by diluting luciferase (EC 1.13.12.7) at 3 .mu.g/mL in 0.1M Tris-acetate pH 7.7, 2 mM EDTA, 10 mM magnesium acetate, 0.1% BSA, 1 mM dithiothreitol, 0.4 mg/mL PVP, and 0.4 mM D-luciferin. A 5 .mu.L of reaction solution has been added to a well of 384-well plate and the luminescence intensity vs. time was recorded on Orion II microplate luminometer at 25° C. (Berthold detection Systems (Pforzheim, Germany)). A first derivative, ∂l(t)l∂t, for the luminescence intensity has been calculated. The derivative was calculated by selecting a group of three or more consecutive measurements of the luminescence intensity and calculating by a least square method the “instant” slope of the luminescent intensity which said slope represents the first derivative of the luminescence at the time when the respective measurements were made. The average intensity for the respective group, l(t), was calculated and the two values for the group, i.e., the slope ∂l(t)l ∂t and l(t), were recorded. Then a next group of consecutive measurements was selected and processed for ∂l(t)l ∂t and l(t), and so forth until all available data were processed. A plot of ∂l(t)l∂t vs. l(t) for all processed data was constructed as shown in panel B. The luminescence intensity at this assay conditions is directly proportional to the concentration of dATP such that l(t) can be expressed in dATP concentration units shown in Panel B.

### Example 2

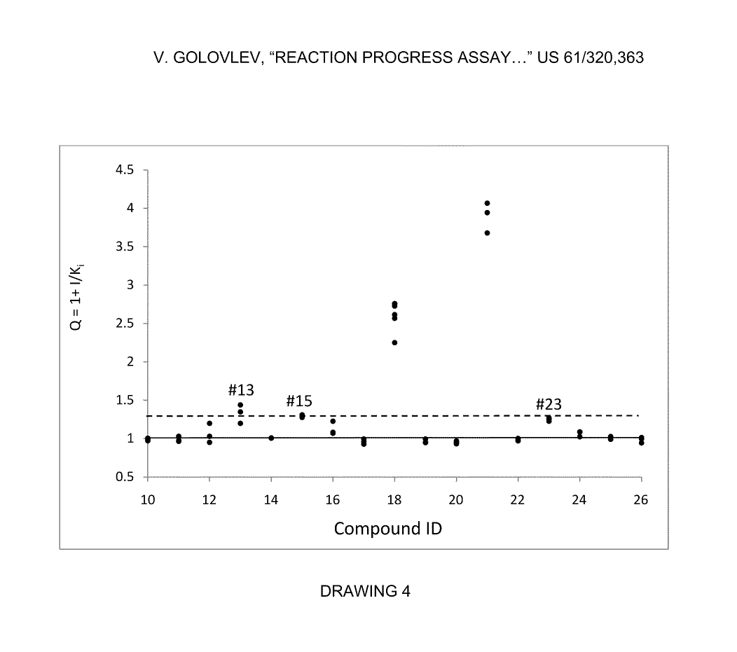

Panel D of the Drawing 3 shows an example of luminescence vs. time captured for the primary reaction of cDNA synthesis carried out by HIV-I RNA-dependent DNA polymerase (reverse transcriptase) in presence of MT-4S allosteric inhibitor (CAS 147127-20-6, U.S. Pat. No. 6,353,112, incorporated herein by reference). The reaction was set by adding: 16 .mu.L of RNAse free water; 2 .mu.L of 10× reaction buffer (400 mM Tris-HCl, 60 mM MgCl.sub.2, 100 mM Dithiothreitol, 20 mM spermidine, pH 7.9 @ 25° C.); 2 .mu.L of template, polyuridylic acid at 1 .mu.g/.mu.L (Midland Certified Reagent Company, Cat. No. P-3004); 2 .mu.L of primer poly(A).sub.18 at 0.5 .mu.g/.mu.L (Polydeoxyadenylate, 18 base long purchased from Integrated DNA technologies); 20 .mu.M of dATP; 3 U of HIV-I (Worthington, Cat. No. LS05006); 3 .mu.L of ATP detection solution prepared according to vendor's protocol (Sigma-Aldrich, Cat. No. FLAA). Concentration of the inhibitor: (D1) 0 nM; (D2) 1.2 nM; (D3) 3.7 nM. The (D4) is the reference with no HIV-1 transcriptase added. With increasing concentration of inhibitor the activity of HIV-I polymerase is decreasing as indicated by lower rate of decline of luminescence signal vs. time. Data in FIG. 4 panel D have been used to measure inhibition constant for MT-4S by analyzing data as disclosed by method of present invention. The inhibition constant for MT-4S found by assay of this invention: Kᵢ=0.9±0.15 nM.

### Example 3

The method of present invention has been demonstrated for detection of weakly binding inhibitors for subsequent use in Fragment-Based Drug Discovery (FBDD). A set of a reference (no inhibitor) and 25 compounds has been selected, where selected compounds have a high similarity to a group of inhibitors that include pentciclovir, entecavir, ganciclovir, and valaciclovir. The compounds have been identified by Tanimoto similarity score with 80% cut off retrieved from NCBI/PubChem (PubChem IDs: Blank, CID23361, CID5429, CID764, CID967, CID1054, CID1676, CID5869315, CID5779, CID4045, CID795, CID1088, CID2153, CID546, CID5071, CID790, CID4421, CID3657, CID3415, CID2130, CID66068, CID71655, CID1088, CID65059, CID2427810324, and CID439750). The HIV-I polymerase (Worthington Biochem., Cat. No. LS05006) was screened for inhibition by compounds at concentrations of the respective inhibitor compounds of 0.5 mM and assay condition as described in Example 1 herein above. In Drawing 4 the inhibition efficiency of the compounds is presented by Q=1+[I]/K.sub.i, where [I] is the concentration of the inhibitor, and K.sub.i is the inhibition coefficient. As illustrated in Drawing 4, the assay of the present invention has the sensitivity for detection of inhibitors having inhibition constant about 2,000 .mu.M, as illustrated by compound #13, #15, and #23 on the Drawing 4.

### Example 4

In this example the method of present invention was used for continuous monitoring of phosphorylation reaction carried out by p38 catalyzed phosphorylation of ATF-2 fusion protein (Cell Signaling, Cat. No. #9224). The reaction was started by adding 300 ng each of p38α and ATF-2 (CellSignaling, Cat. Nos. #7474, and #9224) to 15 .mu.L of 15 mM HEPES buffer (pH=7.4) with added 15 mM MgCl.sub.2; 6 mM Mn Cl.sub.2; and 30 pmole ATP. The luminescence reaction was started by adding 3 .mu.L of luciferin-luciferase ATP detection solution (Cat. No. FLAA, Sigma-Aldrich) to 15 .mu.L of the reaction mix. A 6 .mu.L of the assay solution was placed to a well of 384-well plate and luminescence was recorded vs. time. The reaction was carried out with and without substances known to modulate activity of p38α. Data where analyzed by constructing Michaelis-Menten plot as disclosed in Example 1 or by analyzing the luminescence intensity vs. time as disclosed in Example 2. The assay was able to detect weak inhibitors of p38α with K.sub.i up to 1 mM.

The luminescent intensity in Examples 1-4 was recorded on microplate luminometer Orion II from Berthold Detection Systems (Pforzheim, Germany). It is appreciated that other instruments for recording luminescence can be used. One example, given by way of illustration, and not bay way of limitation is Alliance XD-79LS-26MX cooled CCD system with a 4.7 megapixel 16-bit camera distributed by Aurogene (Knoxyille, Tenn.). The advantage of CCD-based instruments is in the superior throughput capabilities for recording luminescence from all wells simultaneously when using 96-, 384- and 1536-well plates. Data analysis techniques can be used for quantitation of luminescence from individual wells and recording luminescence vs. time by analyzing a continuous stream of images acquired by CCD system as the enzymatic reaction proceeds. The acquired data subsequently can be analyzed for measurement enzyme activity and for identification of substance capable of modulating activity of enzymes of interest as disclosed in the method of present invention.

We claim:

1. A method for continuous monitoring enzymatic reaction and measuring activity of a target enzyme, comprising: 1) performing two enzymatic reactions, the primary reaction catalyzed by a target enzyme, and the secondary detection reaction catalyzed by a detection enzyme, both reactions performed at the same time in the same reaction volume and wherein the primary and secondary enzymatic reaction consumes the same substrate; 2) maintaining reaction conditions at which a substantial amount of substrate is consumed by the primary enzymatic reaction during the time of monitoring the enzymatic reaction, wherein the amount of substrate consumed is at least 10% and most preferably is more than 90% of the amount of substrate present at the time the primary enzymatic reaction started; 3) quantifying and recording vs. time the products or physical effects produced by detection enzymatic reaction, wherein the products or physical effects are proportional to the amount of substrate available; 4) analyzing data for the amount of substrate vs. time and constructing various plots for the reaction rate vs. substrate amount or, equally acceptable, by directly analyzing the data for the amount of substrate vs. time; 5) measuring the target enzyme activity and reaction constant by analyzing data for the substrate vs. time or by analyzing data for the rate of reaction vs. the amount of substrate.

2. The method of claim 1, wherein the target enzyme is from a group comprising NTP- and dNTP-dependent enzymes.

3. The method of claim 1, wherein the target enzyme is from a group comprising DNA-dependent and RNA-dependent polymerases.

4. The method of claim 1, wherein the target enzyme is from a group comprising protein phosphotransferase (EC 2.7.1.37).

5. The method of claim 1, wherein the target enzyme is from a group comprising ATPases, helicases, topoisomerases, and synthases.

6. The method of claim 1, wherein the detection enzyme is a luciferase (EC 1.13.12.7).

7. The method of claim 1, wherein the physical effect is the emission of luminescent light.

8. The method of claim 1, wherein the detection enzyme product is a substance with a desirable fluorescence or light-absorbing properties.

9. The method of claim 1, wherein analyzing data comprising a calculation of time derivative of the amplitude of the physical effect vs. time.

10. The method of claim 1, wherein analyzing data comprising construction trend line, measuring intersection points of the trend lines and axes of respective plot and applying formulas for calculating enzyme activity and reaction constant, said formulas linking the crossing points, the target enzyme reaction constant, the target enzyme activity and substrate concentration.

11. A method for continuous monitoring enzymatic reaction and measuring activity of a target enzyme in presence of a substance that modulating the activity of target enzyme, comprising: 1) performing two enzymatic reactions, the primary reaction catalyzed by a target enzyme, and the secondary detection reaction catalyzed by a detection enzyme, both reactions performed at the same time in the same reaction volume in presence of the compound modulating the target enzyme activity and wherein the primary and secondary enzymatic reaction consumes the same substrate; 2) maintaining reaction conditions at which a substantial amount of substrate is consumed by the primary enzymatic reaction during the time of monitoring the enzymatic reaction, wherein the amount of substrate consumed is at least 10% and most preferably is more than 90% of the amount of substrate present at the time the primary enzymatic reaction started; 3) quantifying and recording vs. time the products or physical effects produced by detection enzymatic reaction, wherein the products or physical effects are proportional to the amount of substrate available; 4) analyzing data for the amount of substrate vs. time and constructing various plots for reaction rate vs. substrate amount or, equally acceptable, by directly analyzing the data for the amount of substrate vs. time; 5) measuring activity of the target enzyme and reaction constant for the target enzyme in presence of the compound that modulates the activity of target enzyme.

12. The method of claim 11, wherein the method is used for screening a library of compounds.

13. The method of claim 11, wherein the method is used for screening and identifying compounds that modulating the target enzyme activity.

14. The method of claim 11, wherein the target enzyme is from a group comprising NTP- and dNTP-dependent enzymes.

15. The method of claim 11, wherein the physical effect is emission of luminescent light.

16. The method of claim 11, wherein the detection enzyme is a luciferase (EC 1.13.12.7).

17. A method for analyzing data and measuring activity of a target enzyme comprising: 1) recording vs. time the decline of the amount of substrate as the substrate is consumed by enzymatic reaction catalyzed by said target enzyme, and wherein the decline of substrate is measured by detecting a product or physical effect produced by a detection enzymatic reaction; 2) constructing a semi-logarithmic plot of said amount of product or physical effect vs. time, and wherein the amount of said product or physical effect is plotted on logarithmic scale vs. a linear time scale; 3) constructing a trend line, wherein the trend line approximates the decline of said product or physical effect vs. time at the portion of the plot that is closely match a simple exponential decline; 4) measuring the points in which the trend line crosses the vertical axis t=0, which represents the moment of time when the recording started, and the horizontal axis that represents the respective substrate amount at t=0; 5) calculating the enzyme activity and reaction constant according to the formulas that link the crossing points, the target enzyme reaction constant, the target enzyme activity and substrate concentration available at t=0.

18. The method of claim 17, wherein the amount of product or physical effect vs. time is recorded in presence of compound that modulates activity of the target enzyme.

19. The method of claim 18, wherein data analysis is repeated for a plurality of recorded data sets, each set representing one compound, for screening a library of compounds and identifying compounds capable of modulating activity of the target enzyme.

20. A method for analyzing data and measuring activity of a target enzyme comprising: 1) recording vs. time the decline of the amount of substrate as the substrate is consumed by target enzymatic reaction, and wherein the decline of substrate is measured by detecting a product or physical effect produced by a detection enzymatic reaction; 2) calculating a time derivative of the amount of product or physical effect vs. time, wherein the product or physical effect is produced by detection enzymatic reaction; 3) constructing a plot of said time derivative vs. the product or physical effect produced by detection enzymatic reaction; 5) calculating the enzyme activity and reaction constant by applying various models for the rate of enzymatic reaction vs. the amount of reaction substrate.
